SEQUENCE LISTING (10 sequences)

SEQ ID NO: 1 - DNA, 294 bp, synthetic construct
  source 1..294: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaa

SEQ ID NO: 2 - DNA, 78 bp, synthetic construct
  source 1..78: mol_type = other DNA
ggcatcggtgcggttttgaaggtattaacgacgggtcttccggctttaattagttggattaaacgtaagcggcagcag

SEQ ID NO: 3 - DNA, 957 bp, synthetic construct
  source 1..957: mol_type = other DNA
aggaggtttgatcctatgggaacaatgcatacatccgagttgctaaaacacatttatgacatcaatttgtcatatttactccttgcacagcgtttgatcgtccaggacaaagcatctgcgatgttccgcctcggtatcaacgaagagatggcaaacacactgggcgcgttgaccctgccgcagatggtcaaactggcggagacgaaccagttagtttgtcatttccggtttgacgatcatcagacgatcacccgtttgactcaggattcgcgcgtcgatgacttacagcagattcacacaggtatcatgctttcaacgcgtctgctcaatgaagtggacgatacggcgcgtaagaaaagggcatgaaagggcatgataatgagtgaaaaaagcattgttcaggaagctcgcgatatccagttggcgatggagttgattaatcttggcgctcgtctacaaatgctggaaagcgaaacacagctcagccgtggtcgcctcatcaggctgtacaaagaattacgcggtagcccgccgcctaaagggatgctgccattttcgacagactggtttatgacctgggagcaaaatattcatgcctccatgttctgcaacgcctggcaatttttactgaagaccggcttatgcagcggtgtggatgcggtgattaaagcttatcggctttatcttgagcagtgtccgcaaccgcctgaagggccgttgttggcgctgactcgcgcatggacgctggtgcgttttgttgaaagtgggttgcttgaattgtcgagctgtaactgctgcggtgggaactttattacccatgcgcatcagcccgtaggcagctttgcgtgtagtttatgccagccgccatcccgcgcagtaaaaagacgtaaactttcccgagatgctgccgatattattccacaactgctggatgaacagatcgaacaggctgtttaa

SEQ ID NO: 4 - DNA, 78 bp, synthetic construct
  source 1..78: mol_type = other DNA
ggcggcagcagccatcaccatcaccatcacagcagcggcggccgcgtgaaacgccgcgtgaaacgccgcgtgaaacgc

SEQ ID NO: 5 - DNA, 42 bp, synthetic construct
  source 1..42: mol_type = other DNA
ggcggcagcagccatcaccatcaccatcacagcagcggcggc

SEQ ID NO: 6 - DNA, 485 bp, synthetic construct
  source 1..485: mol_type = other DNA
gctagcaggaggtttgacctatgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagccatcaccatcaccatcacagcagcggcggccgcgtgaaacgccgcgtgaaacgccgcgtgaaacgcatgggcatcggtgcggttttgaaggtattaacgacgggtcttccggctttaattagttggattagtcgtaaaaaacgtcagcagtaagagctc

SEQ ID NO: 7 - DNA, 446 bp, synthetic construct
  source 1..446: mol_type = other DNA
gctagcaggaggtttgacctatgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagccatcaccatcaccatcacagcagcggcggcatgggcatcggtgcggttttgaaggtattaacgacgggtcttccggctttaattagttggattaaacgtaagcggcagcagtaagagctc

SEQ ID NO: 8 - DNA, 58 bp, synthetic construct
  source 1..58: mol_type = other DNA
gatcgatcgagctcaggaggtttgatcctatgggaacaatgcatacatccgagttgct

SEQ ID NO: 9 - DNA, 49 bp, synthetic construct
  source 1..49: mol_type = other DNA
gatcgatcgtcgacttaaacagcctgttcgatctgttcatccagcagtt

SEQ ID NO: 10 - DNA, 1107 bp, synthetic construct
  source 1..1107: mol_type = other DNA
atgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtag

(19) United States
(12) Patent Application Publication
(10) Pub. No.: US 2026/0022390 A1
(43) Pub. Date: Jan. 22, 2026

(54) RECOMBINANT EXPRESSION VECTOR FOR MELITTIN SECRETION AND ATTENUATED SALMONELLA STRAIN TRANSFORMED THEREWITH

(71) Applicant: THE INDUSTRY & ACADEMIC COOPERATION IN CHUNGNAM NATIONAL UNIVERSITY (IAC), Daejeon (KR)

(72) Inventors: Hyo Jin LEE, Daejeon (KR); Heung Jin JEON, Daejeon (KR); Solbi KIM, Daejeon (KR); Eunji KIM, Daejeon (KR); Minju HAN, Chungcheongbuk-do (KR); Myung-won LEE, Sejong (KR); Sora KANG, Daejeon (KR)

(73) Assignee: THE INDUSTRY & ACADEMIC COOPERATION IN CHUNGNAM NATIONAL UNIVERSITY (IAC), Daejeon (KR)

(21) Appl. No.: 18/997,452

(22) PCT Filed: Jul. 6, 2023
(86) PCT No.: PCT/KR2023/009603

§ 371 Date: Jan. 21, 2025

(30) Foreign Application Priority Data
Jul. 21, 2022 (KR) 10-2022-0090611

(51) Int. Cl. C12N 15/74; A61K 35/74; A61P 35/00; C07K 14/255; C07K 14/435

(52) U.S. Cl. CPC C12N 15/74; A61K 35/74; A61P 35/00; C07K 14/255; C07K 14/43572

(57) ABSTRACT

The present disclosure relates to a recombinant expression vector for secretion of melittin and an attenuated Salmonella strain transformed therewith, and provides an attenuated Salmonella strain transformed with a recombinant expression vector including an flgM gene and a melittin gene, an anticancer pharmaceutical composition including the attenuated Salmonella strain as an active ingredient, and a recombinant expression vector capable of improving the secretion ability of melittin.

### TECHNICAL FIELD

The present disclosure relates to a recombinant expression vector for the secretion of melittin and an attenuated Salmonella strain transformed therewith. The present application claims priority to Korean Patent Application No. 10-2022-0090611, filed on Jul. 21, 2022, with the Korean Intellectual Property Office, the disclosure of which is incorporated in the present specification by reference.

### BACKGROUND ART

Cancer is a disease that ultimately threatens life as cells continue to proliferate, invade surrounding tissues, and destroy normal cells. As the human body's regulatory ability declines with aging, the body becomes more vulnerable to cancer, and in an aging society, cancer remains the number one cause of death overall, more than double the mortality rate of heart disease, which ranks second.

The most effective response system for treating disease is to strengthen the immune system, but cancer cells are not foreign invaders, so they evade the body's immune response without triggering it. Certain viruses or bacteria are more likely to be infected by cancer cells than normal cells, and infected bacteria may become targets of immune cell attack. Accordingly, anticancer treatment methods have been proposed that deliberately infect specific viruses or bacteria to stimulate the body's immune response to fight cancer cells. Infectious bacteria such as Shigella, Vibrio cholerae, and pathogenic E. coli only invade the cells of the intestinal tract, but do not reach the liver and spleen, which are important organs for triggering the immune response. In contrast, Salmonella may invade the spleen and liver through the lymph nodes and stimulate a systemic immune response. Specifically, Leschner et al. (J. Mol. Med. 2010, 88, 763-773) infected mice bearing CT26 tumors intravenously with fluorescent Salmonella and tracked the route of infection over time. As a result, it was shown that immediately after infection, the whole body was infected through the blood of the mouse, and 20 minutes after infection, Salmonella accumulated in the spleen and liver, and 24 hours later, Salmonella was observed to have concentrated accumulation only in the tumor tissue.

However, Salmonella is a representative bacteria that causes food poisoning and may cause sepsis through infection, which is life-threatening, making Salmonella too pathogenic to be used directly in cancer treatment. A research team at Yale University in the United States announced that if Salmonella is genetically modified, Salmonella may be attenuated by removing only the toxicity while maintaining the tumor-attacking characteristic, and that tumors may be suppressed by inducing immune stimulation through injection of the attenuated Salmonella. However, attenuated Salmonella have low survivability and reduced immune-inducing ability, and there is a concern that mutations may cause the attenuated strain to transform into wild-type Salmonella, which may lead to sepsis. In addition, due to the issues with the lack of research and development funds, most anticancer treatments using attenuated Salmonella have been discontinued or put on hold in phase 1 of the clinical process.

The potential for developing sepsis associated with cancer treatment using bacteria such as Salmonella is a critical issue that must be overcome, and in addition, the stimulation of the immune response must also be actively achieved. Accordingly, research is mainly focused on developing strains that may additionally express or suppress substances that are helpful in anticancer treatment along with attenuation and immune activation, and using these strains in anticancer treatment. Korean Patent No. 10-0852687 discloses a Salmonella strain expressing tumor necrosis factor alpha, by transducing a tumor necrosis factor alpha protein vector into an attenuated Salmonella strain, and a composition for anticancer treatment containing the same, and Korean Patent No. 10-1750007 discloses a solid cancer treatment using an attenuated Salmonella strain in which L-asparaginase may be selectively expressed at a tumor site.

The main component of bee venom, “melittin”, is an amphipathic peptide consisting of 26 amino acids, with hydrophobic amino acids distributed in the N-terminal region and hydrophilic amino acids in the C-terminal region. It is known that through this structure of melittin, melittin has membrane-perturbing effects such as pore formation, fusion, and vesicle formation, and melittin pierces the surface or cell membrane of cancer cells to create holes, causing cancer cells to die. In addition, melittin's cell membrane-piercing effect is known to promote the release of cytokines that trigger inflammatory responses.

Accordingly, the inventors of the present disclosure have prepared a genetic construct that links the flgM gene and a gene encoding melittin, and a recombinant strain containing the same, and by introducing the flgM gene, the strain has excellent secretion ability and anticancer effect by enabling the secretion of flgM and melittin through the Type 3 Secretion System, and have completed the present disclosure by having a cancer targeting effect through the cancer targeting ability of the strain.

The information in this Background Art section is intended solely to enhance the understanding of the background of the present disclosure, and may not contain information that constitutes related art, known to a person of ordinary skill in the art to which the present disclosure belongs.

### DISCLOSURE OF INVENTION

### Technical Problem

An objective of the present disclosure is to provide a recombinant expression vector for the effective secretion of a melittin protein, which exhibits excellent anticancer effects, and an attenuated Salmonella strain that has been transformed therewith.

Another objective of the present disclosure is to provide a pharmaceutical composition for anticancer use that includes the attenuated Salmonella strain as an active ingredient.

### Solution to Problem

One aspect is to provide an attenuated Salmonella strain, transformed with a recombinant expression vector including: an flgM gene; and a melittin gene.

In the present specification, “flgM” refers to a protein (or a gene encoding such a protein) that inhibits a σ factor, which is responsible for recruiting RNA polymerase necessary for late flagellar transcription in the type 3 secretion system of bacteria. In the present specification, “type 3 secretion system” refers to a multi-protein complex structure with a syringe-shaped mechanism (Mota L J et al, Ann Med. (2005); 37 (4): 234-249) that injects pathogenic active proteins directly into the cytoplasm through the host cell membrane, a method of delivering pathogenic substances developed in Gram-negative bacteria. In the type 3 secretion system, the flgM protein remains in the cytoplasm during the formation of the hook-basal body, the basic structure capable of forming the flagellum on the cell membrane, and acts as an anti-σ²⁸ factor that inhibits the transcription of class III genes, for example, the flagellin subunit FliC or the stator protein MotAB. Subsequently, after completion of the hook-basal body structure, a change in substrate specificity occurs simultaneously within the flagella secretion system, causing flgM to be secreted out of the cell through the central channel of the hook, and subsequently initiating σ²⁸-dependent transcription within the cell. The secretion mechanism of flgM through the flagellar formation process in the type 3 secretion system is illustrated in FIG. 2.

Specifically, the present disclosure has produced a genetic construct including the flgM gene and the melittin gene, which is a protein to be secreted, to enable the secretion of flgM through the type 3 secretion system of a strain, and secretion of melittin outside the strain.

In the present specification, “melittin” is an amphipathic peptide consisting of 26 amino acids that is a main component of bee venom. In the present specification, the term “bee venom (BV)” refers to a mixture of acidic and basic secretions produced by the abdomen of the honey bee (Apis mellifera), which is a colorless, bitter liquid, the main components of which are the peptides melittin, apamin, and mast cell degranulating (MCD) peptide, as well as the enzyme phospholipase A2 (PLA2), etc. and includes various other trace components. Thus, the melittin of the present disclosure may be, but is not limited to, isolated from the bee venom of the honey bee (Apis mellifera). Melittin has a distribution of hydrophobic amino acids in the N-terminal region and hydrophilic amino acids in the C-terminal region. Due to this structure, melittin has membrane-perturbing effects such as pore formation, fusion, and vesicle formation, and thus penetrates and punctures the surface or cell membrane of cancer cells, thereby killing cancer cells. In addition, melittin has the effect of inducing a secondary anti-cancer immune response by bursting cancer cells and promoting the induction of immune cells. The melittin sequence may be obtained from the NCBI GeneBank, a publicly available database.

In the present specification, the term “gene” should be considered in its broadest sense and may encode a structural protein or a regulatory protein. In this context, the regulatory protein may include a transcription factor, a heat shock protein, or a protein involved in DNA/RNA replication, transcription, and/or translation.

In the present specification, the term “transformation” refers to introducing a vector including a polynucleotide encoding a target protein into a host cell such that the protein encoded by the polynucleotide is expressed in the host cell. For example, the polynucleotide may be introduced into the host cell in the form of an expression cassette or may be introduced into the host cell in its own form and operably linked to a sequence required for expression in the host cell, but is not limited thereto. According to an embodiment, a recombinant expression vector including the flgM gene and a melittin gene, the protein of interest to be secreted, may be prepared and used to transform a Salmonella strain so that the Salmonella strain not only expresses melittin, but also the secretion of melittin outside the cell may be controlled.

In the present specification, the term “attenuated” refers to the artificial attenuation of the virulence of a living pathogen, such as by modifying genes involved in the essential metabolism of the pathogen so that the pathogen is unable to cause disease in the body and only stimulates the immune system to induce immunity. Genes causing attenuation of Salmonella are well known in the art, and for example, the attenuated Salmonella strain may have lost the function of one or more genes selected from the group consisting of aroA, aroC, aroD, aroE, asd, Rpur, htrA, ompR, ompF, ompC, galE, cya, crp, cyp, phoP, phoQ, rfaY, dksA, hupA, sipC, clpB, clpP, clpX, pab, nadA, pncB, pmi, rpsL, hemA, rfc, poxA, galU, cdt, pur, ssa, guaA, guaB, fliD, flgK, flgL, relA, and spoT. In order to prevent reversion to the wild-type and further attenuate virulence during in vivo application, the function of two or more of the above genes may be lost. According to an embodiment, the attenuated Salmonella strain may be a Salmonella strain in which the aroA, aroD, and asd genes have been lost.

The attenuated Salmonella strain has anticancer activity by inducing an immune response targeted to the tumor site. Meanwhile, when transformed with a recombinant expression vector of the present disclosure, the Salmonella strain may exhibit more potent anticancer activity by being targeted to the tumor site and having an enhanced ability to secrete melittin, for example, an flgM-melittin fusion protein.

In an embodiment, the flgM gene and the melittin gene may be linked via a linker. In the present specification, “linker” refers to a polypeptide chain used to link a heterologous domain to a protein of interest, and in an embodiment, the linker may be any one selected from the group consisting of, but not limited to, for example, (GₗSₘ)ₙ(ISm), (GₗSₘ)ₙ(I, m, and n each ≥2), (GₗSₘ)ₙ-Hₚ-(GₗSₘ)ₙ(I, m, and n each ≥1, H≥6), (G₄S)ₐ(EAAAK)_{b}(G₄S)ₐ (a, b is an integer from 1 to 4), (G₄S)ₚ(EAAAK)_{q} (p, q is an integer from 1 to 4), (EAAAK)ₓ(G₄S)_{y} (x, y is an integer from 1 to 4), A(EAAAK)₄ALEA(EAAAK)₄A, (GSSGGS)ᵢ (i is an integer from 1 to 4), KESGSVSSEQLAQFRSLD, EGKSSGSGSESKST, GSAGSAAGSGEF, (EAAAK)ₖ (k is an integer from 1 to 5), CRRRRRRREAEAC, GGGGGGGG, GGGGGG, AEAAAKEAAAAKA, PAPAP, VSQTSKLTRAETVFPDV, PLGLWA, TRHRQPRGWE, AGNRVRRSVG, RRRRRRRRR, GSSGGSGSSGGSGGGGDEADGSRGSQKAGVDE, PAPAP, VSQTSKLTRAETVFPDV, PLGLWA, TRHRQPRGWE, AGNRVRRSVG, RRRRRRRR, and GSSGGSGSSGGSGGGDEADGSRGSQKAGVDE.

The linker may be, for example, a peptide linker of 10 to 16-mer, and the length of the peptide may be a peptide linker of 10 to 15-mer, 10 to 14-mer, 10 to 13-mer, 10 to 12-mer, 10 to 11-mer, 12 to 16-mer, 12 to 15-merm, 12 to 14-mer, 12 to 13-mer, 13 to 16-mer, 13 to 15-merm, or 13 to 14-mer. The length of the linker may affect the level of flgM-melittin fusion protein secretion of the Salmonella strain according to an embodiment.

In an embodiment, the attenuated Salmonella strain may have an enhanced ability to secrete melittin. In an embodiment, the attenuated Salmonella strain may be transformed with a vector including an arabinose-inducible promoter to control the expression of melittin in the presence of arabinose, and flgM of the type 3 secretion system may be used to effectively secrete the melittin protein out of the strain.

In an embodiment, the attenuated Salmonella strain may further include an flhDC gene. The flhDC gene is a primary regulator of the flagellin operon, which may control expression of the flgM gene, and thus including the flhDC gene in the genetic construct may contribute to enhanced expression level or secretion amount of flgM-melittin.

In an embodiment, the attenuated Salmonella strain may be a Salmonella strain deposited under Accession No. KCTC15485BP. According to an embodiment, the attenuated Salmonella strain was transformed by a recombinant expression vector including an flgM gene-linker-melittin-flhDC gene construct, wherein the gene construct was capable of enhancing the secretion ability of flgM-melittin of the attenuated Salmonella strain while retaining the inherent anticancer activity of melittin. Therefore, the transformed attenuated Salmonella strain according to an embodiment may be utilized as an active ingredient for the treatment of cancer.

Another aspect provides an anti-cancer pharmaceutical composition or a pharmaceutical composition for the prevention or treatment of cancer including the attenuated Salmonella strain as an active ingredient.

In the anticancer pharmaceutical composition or pharmaceutical composition for the prevention or treatment of cancer, the same terms or elements mentioned above as those already mentioned are as described above.

The term “cancer”, the disease to be prevented or treated by the compositions of the present disclosure, is meant collectively for diseases caused by cells having aggressive characteristics, in which the cells divide and grow in defiance of normal growth limits, invasive characteristics, in which the cells penetrate surrounding tissues, and metastatic characteristics, in which the cells spread to other parts of the body. The cancer may be, for example, a solid tumor, preferably a tumor including cancer tissue or cancer cells in which the cancer-targeting ability of the Salmonella strain may be exerted, and thus the application may be expanded without limitation. For example, the type of cancer may be any one selected from the group consisting of, pancreatic cancer, colon cancer, stomach cancer, liver cancer, colorectal cancer, brain cancer, breast cancer, thyroid cancer, bladder cancer, esophageal cancer, uterine cancer, and lung cancer, but is not limited thereto.

The composition of the present disclosure may be prepared by a method known in the pharmaceutical field for use as an anticancer agent, and may be used on its own or mixed with a pharmaceutically acceptable carrier, forming agent, diluent, etc.

In the present specification, a “pharmaceutically acceptable carrier” may be determined in part by the particular composition being administered, as well as in part by the particular method used to administer the composition. Accordingly, suitable formulations of the compositions are very diverse, for example, the compositions may be formulated for parenteral (in other words, intramuscular, intradermal, or subcutaneous) administration or nasopharyngeal (in other words, intranasal) administration. Generally, parenteral dosage forms include sterile aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers may include water, alcohol/aqueous solutions, emulsions, or suspensions, including brines and buffered media. Parenteral vehicles may include sodium chloride solution, Ringer's dextrose, dextrose and sodium chloride, lactated Ringer's, or fixed oils. Preservatives and other additives may also be present, for example, as antimicrobial agents, antioxidants, chelating agents, and inert gases, etc.

The dosage of the active ingredient according to the present disclosure may be appropriately selected depending on the absorption of the active ingredient in the body, the form of the preparation, the age, sex and condition of the patient, the severity of the symptoms, etc. and may be administered once daily or in several divided doses. The typical dosage is 0.001 mg/kg day to 10 g/kg·day.

Another aspect provides a method of preventing or treating cancer, including administering to a subject an anticancer pharmaceutical composition including the attenuated Salmonella strain as an active ingredient in an amount effective to prevent or treat cancer. In the method of preventing or treating cancer, any of the terms or elements mentioned are the same as already mentioned.

The subject may be a mammal. The mammal may be a human, a dog, a cat, a cow, a goat, or a pig.

The administration may be done via any common route as long as the composition may reach the target tissue. For example, the composition may be administered through routes such as eye drop administration, intraperitoneal administration, intravenous administration, intramuscular administration, subcutaneous administration, intradermal administration, transdermal patch administration, oral administration, intranasal administration, intrapulmonary administration, and rectal administration, etc. and specifically, the composition may be administered according to the purpose through a route such as eye drop administration, etc.

In the present specification, the terms “treatment” or “treating” or “alleviating” or “improving” are used interchangeably. These terms refer to methods of obtaining beneficial or desired results, including but not limited to therapeutic benefits and/or prophylactic benefits. Therapeutic benefit refers to any therapeutically significant improvement in, or effect on, one or more diseases, conditions, or symptoms under treatment. In a preventive benefit, the composition may be administered to a subject at risk of developing a particular disease, condition or symptom, or to a subject reporting one or more physiological symptoms of a disease, even if the disease, condition or symptom may not yet be present.

In the present specification, the term, “effective amount” or “therapeutically effective amount” refers to an amount of an agent sufficient to cause a beneficial or desired result. The therapeutically effective amount may vary depending on one or more of the subject and condition being treated, the weight and age of the subject, the severity of the condition, the method of administration, etc. and this may be readily determined by a person skilled in the art. Additionally, the term applies to a dose to provide an image for detection by any of the imaging methods described herein. The specific dosage may vary depending on one or more of the specific agent selected, the dosing regimen followed, whether the composition is administered in combination with other compounds, the timing of administration, the tissue being imaged, and the body delivery system through which the composition is delivered.

Another aspect provides a genetic construct including an flgM gene; and a melittin gene; or a recombinant expression vector including an flgM gene; and a melittin gene.

In the genetic construct or recombinant expression vector, any of the terms or elements mentioned are the same as already mentioned.

In the present specification, the term “genetic construct” refers to an aggregate, functional unit, or construct including the genetic information of a protein of interest, and may be interpreted in the broadest sense. For the purpose of the present disclosure, the genetic construct may refer to, for example, a DNA insert in which an flgM gene, a peptide linker of 10 to 16 mer, and a melittin gene are operably linked, for enhancing the secretion of an flgM-melittin fusion protein from an attenuated Salmonella strain.

In the present specification, the term “vector” refers to a DNA construct containing a nucleic acid sequence encoding a desired polynucleotide operably linked to suitable regulatory sequences so as to enable expression of the desired polynucleotide in a suitable host. The regulatory sequence may include a promoter capable of initiating transcription, any operator sequence for regulating such transcription, a sequence encoding a suitable mRNA ribosome binding site, and a sequence controlling the termination of transcription and translation. After being transformed into a suitable host, the vector may replicate or function independently of the host genome, or may be integrated into the genome itself. The vector may be any one capable of replicating within a host cell. For example, the vector may be a plasmid, cosmid, virus, or bacteriophage in its natural state or recombinant state. The vector may include a selection marker for selecting a host cell containing the vector. The selection markers are for selecting cells transformed with the vector, and markers that confer a selectable phenotype, such as drug resistance, nutrient requirement, resistance to cytotoxic agents, or expression of surface proteins, may be used. For example, the markers may be one or more selection markers selected from the group consisting of ampicillin, neomycin, puromycin, hygromycin, and zeocin. The vector may include a gene involved in replication and/or copy number control, such as a replication origin, a promoter, etc. and may include, but is not limited to, a restriction enzyme site, etc.

In an embodiment, the recombinant expression vector may be operably linked to an inducible promoter. In the present specification, an “inducible promoter” refers to a promoter capable of switching the operation of a gene linked by an inducer, such as, for example, an ara promoter, a tac promoter, a lac promoter, a lacUV5 promoter, a lpp promoter, a pLA promoter, a pRA promoter, a rac5 promoter, an amp promoter, a recA promoter, an SP6 promoter, a trp promoter, a T7 promoter, pBAD promoter, Tet promoter, trc promoter, pepT promoter, sulA promoter, pol 11 (dinA) promoter, ruv promoter, uvrA promoter, uvrB promoter, uvrD promoter, umuDC promoter, lexA promoter, cea promoter, caa promoter, recN promoter, pagC promoter, hip promoter, ansB promoter, or pflE promoter.

In the present specification, “operably linked” may refer to nucleotide sequences being linked on a single nucleic acid fragment such that the function of one is affected by the other. In one specific example, a recombinant expression vector of flgM-(linker)-melittin-pBAD18, in which the gene is operably linked to the ara promoter, was established using the pBAD18 asd plasmid as a vector to express the flgM-melittin protein. The asd gene may consist of a nucleotide sequence of SEQ ID NO: 10.

In an embodiment, a Shine-dalgarno sequence may be inserted at the front end of the genetic construct of the present disclosure to improve the expression efficiency of the melittin. Furthermore, appropriate restriction enzymes may be placed at the front and back ends of the genetic construct of the present disclosure to allow the genetic construct of the present disclosure to be introduced into a vector.

In an embodiment, the flgM gene may be consisted of a nucleotide sequence of SEQ ID NO: 1, and/or the melittin gene may consist of a nucleotide sequence of SEQ ID NO: 2. In addition, the recombinant expression vector may additionally include an flhDC gene, and the flhDC gene may consist of a nucleotide sequence of SEQ ID NO: 3.

In the present specification, “variants” refers to entities that exhibit significant structural identity with a reference entity (for example, a wild-type sequence) but are structurally different from the reference entity in one or more aspects. Whether a particular entity is appropriately considered a “variant” of a reference entity is based on the degree of structural or functional identity with the reference entity. Any biological or chemical reference entity has certain unique structural elements, and a variant, by definition, refers to a distinct chemical entity that shares one or more of those unique structural elements. For example, a polynucleotide may have a unique sequence element including a plurality of nucleotide residues having designated positions relative to each other in linear or three-dimensional space. For example, a polynucleotide may differ from a reference polynucleotide due to one or more differences in nucleotide sequence and/or one or more differences in chemical moieties (for example, carbohydrates, lipids, etc.) covalently bonded to the polynucleotide backbone. In some embodiments, the variant exhibits at least 60%, 65%, 70%, 75%, 80%, 85%, 86%, 87%, 88%, 89%, 90%, 91%, 92%, 93%, 94%, 95%, 96%, 97%, 98%, or 99%, overall sequence identity to the reference polynucleotide.

In an embodiment, the flgM gene; the melittin gene; and the flhDC gene may be whose expression is controlled by the same inducible promoter. The expression of the flgM gene; the melittin gene; and the flhDC gene may be controlled by the same inducible promoter, thereby enhancing the secretion ability of the flgM and melittin proteins outside the strain, and enhancing the anticancer effect of a composition including the strain.

### Advantageous Effects of Invention

The attenuated Salmonella strain according to the present disclosure has been transformed by a recombinant expression vector including an flgM gene-linker-melittin-flhDC gene construct, which may induce expression and secretion of high levels of melittin. Therefore, the recombinant expression vector according to an aspect, the attenuated Salmonella strain transformed with the vector, and the anticancer pharmaceutical composition including the same may be utilized to treat cancer.

### BRIEF DESCRIPTION OF DRAWINGS

FIG. 1 is a diagram schematically illustrating a process of flgM-melittin protein secretion using a type 3 secretion system.

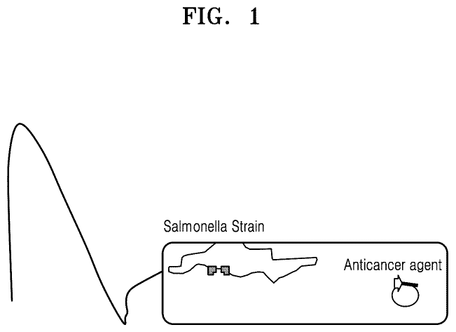

FIG. 2 is a diagram briefly illustrating a secretion mechanism of flgM through an flagellar formation process within the type 3 secretion system.

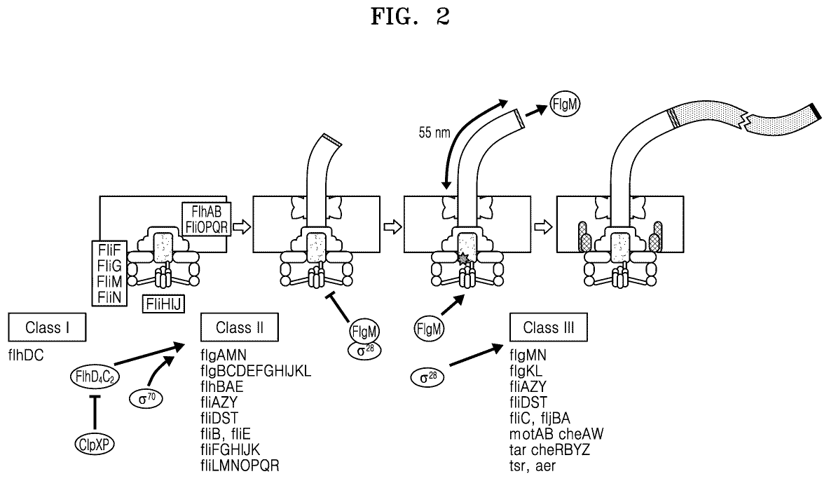

FIG. 3 is a diagram illustrating an open reading frame of an flgM-melittin-flhDC-pBAD18 plasmid.

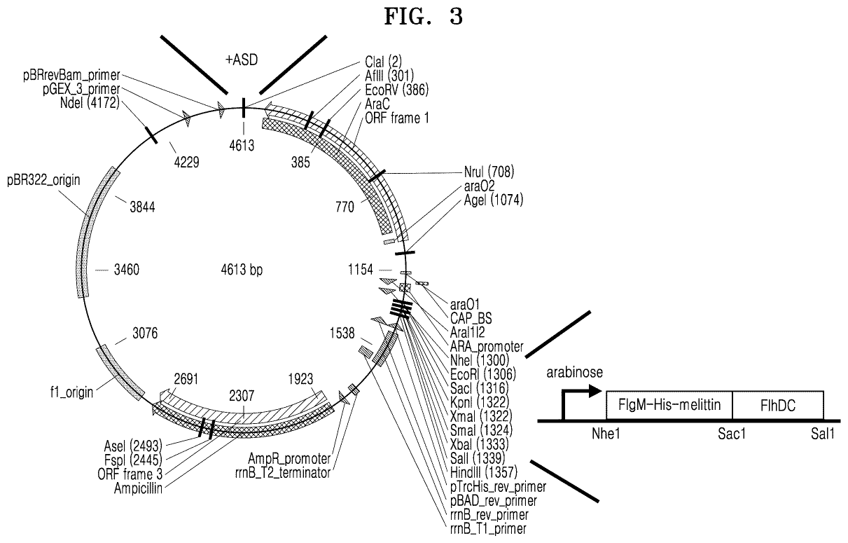

FIG. 4 shows the results of confirming a protein secretion level of an attenuated Salmonella strain transformed using the flgM-linker-melittin-flhDC-pBAD18 plasmid according to an embodiment. Lanes 1 and 2 are the group in which the strain transformed with the flgM-linker-melittin-flhDC pBAD18 plasmid (flgM-linker-melittin-flhDC pBAD18/aroA aro D asd-) was grown for 4 hours, lanes 3 and 4 are the group in which the strain was grown for 6 hours, and lanes 2 and 4, unlike lanes 1 and 3, are the group in which expression was induced by treatment with arabinose.

FIG. 5 shows the results of confirming flgM-melittin expression and secretion levels depending on the type of linker used. Lane 1 uses a linker with SEQ ID NO: 5 and, unlike lanes 2 and 3, is a group that was not treated with arabinose. Lane 2 is the group using the linker with SEQ ID NO: 4. Lane 3 is the group using the linker with SEQ ID NO: 5.

FIG. 6 shows the results of confirming apoptosis through CCK assay in HT-29 and SW480 human colorectal cancer cell lines. (−) is the result of the group not treated with arabinose, (1) is the result of the group applied with the linker of SEQ ID NO: 4, and (2) is the result of the group applied with the linker of SEQ ID NO: 5.

FIG. 7 shows the results of confirming apoptosis through LDH assay in HT-29 and SW480 human colorectal cancer cell lines. (−) is the result of the group not treated with arabinose, (1) is the result of the group applied with the linker of SEQ ID NO: 4, and (2) is the result of the group applied with the linker of SEQ ID NO: 5.

FIG. 8 shows the results of confirming the anticancer effect by treating a mouse breast cancer cell line in vitro using secreted flgM-melittin. (a) of FIG. 8 shows the results of the group that was not treated with anything, (b) of FIG. 8 shows the results of the group that was not treated with arabinose, and (c) of FIG. 8 shows the results of the experimental group that was treated with arabinose to induce expression.

FIG. 9 shows the results of confirming apoptosis through a CCK assay in the 4T1 mouse breast cancer cell line. The results are for the group not treated with arabinose (−) and the group treated with arabinose (+).

FIG. 10 shows the results of confirming apoptosis through CCK assay in the CT26 mouse colorectal cancer cell line. The results are for the group not treated with arabinose (−) and the group treated with arabinose (+).

### MODE FOR THE INVENTION

Hereinafter, preferred examples are presented to aid in the understanding of the present disclosure. However, the following examples are provided only to aid understanding of the present disclosure, and the present disclosure is not limited by the following examples.

### Example 1: Preparation of Plasmid for Melittin Secretion

1-1. Preparation of Genetic Construct Linking flgM Gene and Melittin Gene

A genetic construct according to an embodiment was prepared by linking an flgM DNA sequence of wild-type Salmonella (flgM anti-sigma-28 factor FlgM [Salmonella enterica subsp. enterica serovar Typhimurium str. LT2] Gene ID: 1252690, Locus tagSTM1172 NC_003197.2 (1257036 . . . 1257341, complement)) and a DNA sequence obtained by codon optimization for melittin. Specifically, the flgM DNA sequence and the melittin DNA sequence were linked via a linker, and to increase the expression of the gene, a 14-base Shine-dalgarno sequence (AGGAGGTTTGACCT) was inserted upstream of the flgM gene. Additionally, for cloning, an Nhe I site was inserted at the very front, and a Sac I site was inserted after the termination codon of melittin. Meanwhile, the genetic information of the main constructs in this example is as shown in Table 1 below.

TABLE 1

| | | SEQ |
|---|---|---|
| | | ID |
| Name | Sequence Information | NO |
| [symbol] IgM | atgagcattgaccgtacctcacctttgaa | 1 |
| | acccgttagcactgtccagacgcgcgaaa | |
| | ccagcgacacgccggtacaaaaaacgcgt | |
| | caggaaaaaacgtccgccgcgacgagcgc | |
| | cagcgtaacgttaagcgacgcgcaagcga | |
| | agctcatgcagccaggcgtcagcgacatt | |
| | aatatggaacgcgtcgaagcattaaaaac | |
| | ggctatccgtaacggtgagttaaaaatgg | |
| | atacgggaaaaatagcagactcgctcatt | |
| | cgcgaggcgcagagctacttacagagtaa | |
| | aa [symbol] a | |
| melittin | ggcatcggtgcggttttgaaggtattaac | 2 |
| | gacgggtcttccggctttaattagttgga | |
| | ttaaacgtaagcggcagcag | |
| linker 1 | ggcggcagcagccatcaccatcaccatca | 3 |
| | cagcagcggcggccgcgtgaaacgccgcg | |
| | tgaaacgccgcgtgaaacgc | |
| linker 2 | ggcggcagcagccatcaccatcaccatca | 5 |
| | cagcagcggcgggc | |

[symbol]

indicates data missing or illegible when filed

Thereafter, the flgM-melittin construct was reacted at 37° C. for 2 hours using 10U each of Nhe I and Sac I, and the reaction product was electrophoresed, and 400 bp DNA was confirmed with a gel extraction kit to obtain flgM-melittin insert DNA, in other words, the genetic construct for flgM-melittin (flgM-linker1-melittin: SEQ ID NO: 6, flgM-linker2-melittin: SEQ ID NO: 7).

1-2. Preparation of Genetic Construct Including flhDC Gene

Using a genomic DNA of wild-type Salmonella LT2 as a template and the flhD-sac1-F and flhC-Sal1-r primers in Table 2, a PCR product including an flhDC gene was obtained by PCR, and the specific PCR conditions were as follows; 10× buffer 5 μl, 5×Q solution 10 μl, 2 mM dNTPs 7.5 μl, 20 μM flhD-sac1-f primer 2.5 μl, 20 μM flhC-Sal1-r primer 2.5 μl, Taq polymerase 1 μl, 10 ng/μl genomic DNA 2 μl, and water 19.5 μl were mixed and PCR was performed using the Qiagen system under the conditions of 95° C. for 5 minutes, 30 repeated cycles of 94° C. for 30 seconds, 49° C. for 30 seconds, and 72° C. for 1 minute, followed by 72° C. for 10 minutes. 1 μg of PCR fragment purified using a PCR purification kit was reacted with 10 U each of sac I and Sal I at 37° C. for 2 hours, and the reaction product was purified with a PCR purification kit to produce flhDC insert DNA of SEQ ID NO: 3.

TABLE 2

| | | SEQ |
|---|---|---|
| | | ID |
| Name | Sequence Information | NO |
| flhD-SAC1-f | gatcgatcgagctcaggaggtttgat | 8 |
| | cctatgggaacaatgcatacatccga | |
| | gttgct | |
| flhD-SAl1-r | gatcgatcgtcgacttaaacagcctg | 9 |
| | tggcgatctggttcatccagcagtt | |

1-3. Establishment of flgM-Linker-Melittin-flhDC-pBAD18 Plasmid

Using a pBAD18 asd plasmid as a vector, 10 U each of Nhe I and Sac I enzymes were reacted at 37° C. for 2 hours, and the reaction products were purified with a PCR purification kit to obtain the respective vector DNA. The asd gene inserted in the plasmid may consist of a nucleotide sequence of SEQ ID NO: 10. Afterwards, each vector DNA was ligated with the flgM-melittin insert DNA of Example 1-1 at 25° C. for 30 minutes, and transformed into DH5a competent cells. Thereafter, the transformed cells were plated on LB amp solid medium and cultured at 37° C. to select colonies with antibiotic resistance. Six candidates of the selected colonies were selected and reacted with 10 U each of Nhe I and Sac I at 37° C. for 1 hour, and the production of a 400 bp band was confirmed through electrophoresis. Thereafter, nucleic acid sequence analysis of the candidate group was performed on the plasmids prepared using pBAD18, thereby confirming the insertion of the flgM, linker1, linker2, and melittin genes, and establishing flgM-linker1-melittin-pBAD18 plasmids and flgM-linker2-melittin-pBAD18 plasmids according to an embodiment.

Afterwards, 1 μg of the above-produced flgM-linker1-melittin-pBAD18 plasmid or flgM-linker2-melittin-pBAD18 plasmid was reacted with 10 U of sac I and Sal I at 37° C. for 2 hours, and the reaction product was purified with a PCR purification kit to complete the vector DNA. Afterwards, each vector DNA was ligated with the flhDC insert DNA of Example 1-2 at 25° C. for 30 minutes, and then DH5a competent cells were transformed. The transformed cells were plated on LB amp solid medium and cultured at 37° C. to select colonies with antibiotic resistance. Six candidates of the selected colonies were selected and reacted with 10 U of sac I and Sal I at 37° C. for 1 hour, and the production of a band was confirmed through electrophoresis. The nucleic acid sequence analysis of the candidate group was performed to confirm the insertion of the flhDC gene, and through this, the flgM-linker1-melittin-flhDC pBAD18 plasmid and the flgM-linker2-melittin-flhDC pBAD18 plasmid according to an embodiment were established.

### Example 2: Preparation of Attenuated Salmonella Strain Secreting flgM-Melittin

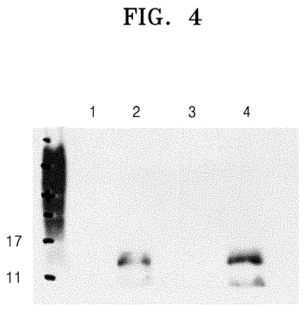

An attenuated Salmonella strain was transformed using the plasmid prepared in Example 1 above. An aro A aro D asd-strain was used as the attenuated Salmonella strain, whereby the attenuated Salmonella strain was engineered to regulate the expression of melittin by arabinose and to secrete melittin out of the cell. A single colony of each of the transformed strains was inoculated into LB amp liquid medium and shake-cultured at 37° C. for 12 to 16 hours. Afterwards, the culture was diluted to OD600 of 0.05 in fresh LB amp liquid medium and shake-cultured at 37° C. for 2 hours. When the OD600 reached 0.4 to 0.6 by shake-culture, arabinose was added to a concentration of 0.2 w % and further shake-cultured at 37° C. for 5 hours. Afterwards, the culture solution was centrifuged at 3000 rpm to separate the supernatant, and the supernatant was filtered through a 0.2 μm filter to completely remove the bacteria. 50 μl of the supernatant from which the bacteria had been removed was mixed with SDS sample buffer and denatured for 5 minutes in a 100° C. heating block, then, centrifuged at 13000 rpm at 4° C. for 5 minutes, and electrophoresed on a 10% polyacrylamide gel. After electrophoresis at 100 V for 1 hour and 30 minutes, Western blotting was performed by staining with Coomassie Brilliant Blue G 250 and destaining with distilled water. Through this, the amount of flgM-melittin in the culture medium was confirmed and shown (FIG. 4). In FIG. 4, Lanes 1 and 2 are the group in which the strain transformed with the flgM-linker-melittin-flhDC pBAD18 plasmid (flgM-linker-melittin-flhDC pBAD18/aroA aro D asd-) was grown for 4 hours, lanes 3 and 4 are the group in which the strain was grown for 6 hours, and lanes 2 and 4, unlike lanes 1 and 3, are the group in which expression was induced by treatment with arabinose.

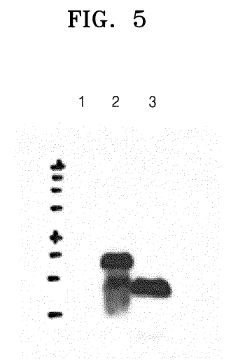

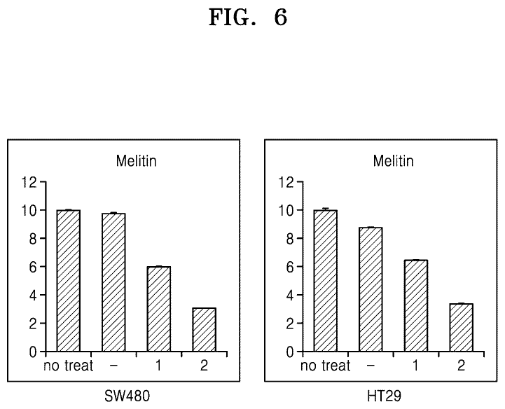

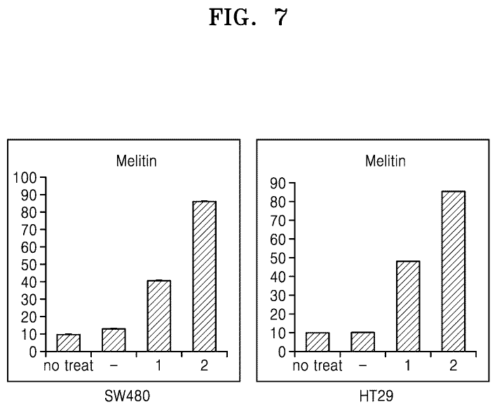

As a result, as shown in FIG. 4, it was confirmed that lanes 2 and 4 treated with arabinose secreted a larger amount of flgM-melittin at the same concentration than lanes 1 and 3 not treated with arabinose. In addition, as shown in FIG. 5, it was confirmed that the secretion amount of flgM-melittin in lane 3 to which a specific linker was applied was superior compared to lane 2. Additionally, as shown in FIGS. 6 and 7, it was confirmed that the level of dead cells was high in the group to which a specific linker was applied. From the above results, it was found that the strain transformed with the flgM-linker2-melittin-flhDC-pBAD18 plasmid according to an embodiment exhibited excellent melittin secretion ability, and in particular, the secretion ability and apoptosis effect of the flgM-linker2-melittin-flhDC pBAD18/aroA aro D asd-strain to which a 14mer linker was applied were very excellent. Accordingly, the inventors of the present disclosure deposited the flgM-linker2-melittin-flhDC pBAD18/aroA aro D asd-strain according to an embodiment at the Biological Resource Center of the Korea Biotechnology Research Institute on Jun. 26, 2023, and received the Accession No. KCTC15485BP (S-flgM-melittin-pBAD18-ASD+/BRD509 asd−).

### Example 3: Testing Antitumor Effect of Melittin In Vitro

3-1. Activity Evaluation of Secreted flgM-Melittin

A single colony of the transformed flgM-linker2-melittin-flhDC pBAD18/aroA aro D asd-strain obtained in Example 2 was inoculated into LB amp liquid medium and shake-cultured at 37° C. for 12 to 16 hours. Afterwards, the culture was diluted to OD600 of 0.05 in fresh LB amp liquid medium and shake-cultured at 37° C. for 2 hours. When the OD600 reached 0.4 to 0.6 by shake-culture, arabinose was added to a concentration of 0.2 w % and further shake-cultured at 37° C. for 5 hours. For comparison, a comparison group not treated with arabinose was shake-cultured under the same conditions. The culture solution was centrifuged at 3000 rpm to separate the supernatant, and the supernatant filtered through a 0.2 μm filter to completely remove the bacteria. Through this, an flgM-melittin fusion protein was obtained.

Meanwhile, 4T1 mouse breast cancer cell line was purchased and each cell was grown in Dulbecco's modified Eagle's medium (DMEM) including 10% fetal bovine serum (FBS) and 1% penicillin-streptomycin, and the cells were seeded in 6-wells when the number of cells was 2×10⁵. After 24 hours, 200 μl of the fusion protein obtained from the supernatant through the above process was treated, cultured for 48 hours, and then observed.

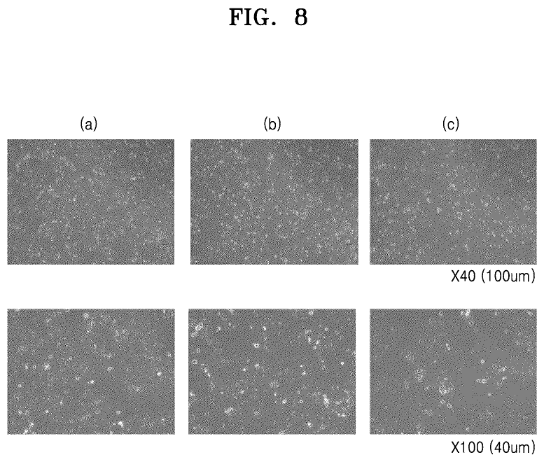

As a result, as shown in FIG. 8, it was confirmed that, the comparison group (b) that was not treated with arabinose showed a decrease in cell level by about 5% compared to the control group (a) that was not treated with anything, whereas the experimental group (c) that was treated with arabinose to induce expression or secretion of flgM-melittin showed a decrease in cell level by 50% or more. 3-2. Evaluation of cancer cell apoptosis induction effect

In order to confirm the effect on inducing cancer cell apoptosis, 4T1 mouse breast cancer cell line, HT-29 and SW480 human colorectal cancer cell lines, and CT26 mouse colorectal cancer cell line were purchased and culture mediums were obtained as in Example 3-1 above, and CCK assay and LDH assay were performed using them. For the CCK assay, 100 μl of the cultured cell line supernatant was transferred to a 96-well plate, 100 μl of CCK solution was added, and then measured with a 450 nm microreader. This experiment was conducted in triplicate to increase reliability. In addition, for the LDH assay, the supernatant of the cultured cell line was centrifuged at 1000 rpm for 3 minutes to remove cell debris, 10 μl of the supernatant and 100 μl of LDH solution were mixed, incubated at room temperature for 30 minutes, and measured using a 450 nm microreader. As with the CCK assay, the experiment was conducted in triplicate.

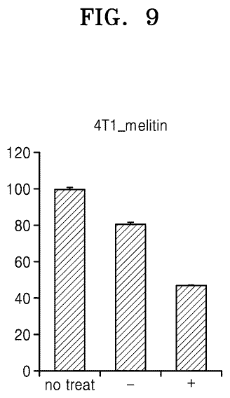

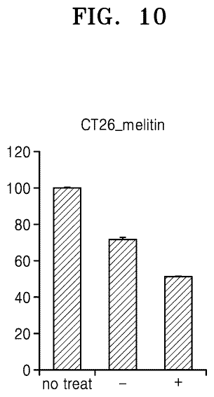

As a result, as shown in FIGS. 6 and 7, it was confirmed that the experimental groups (1,2) that induced expression or secretion of flgM-melittin had the lowest level of detected cellular activity and the highest level of cells that burst and died compared to the comparison group (−) that was not treated or treated with arabinose. Furthermore, as shown in FIGS. 9 and 10, it was confirmed that the experimental group (+) that induced the expression or secretion of flgM-melittin showed the highest level of cells that burst and died compared to the comparison group (−) that was not treated or treated with arabinose. The above experimental results experimentally demonstrate that the transformed attenuated Salmonella strain according to an embodiment possesses the cell killing ability inherent to melittin, while its secretion ability is enhanced through a unique structure linked to flgM, a type 3 secretion system.

While certain aspects of the present disclosure have been described in detail above, it will be apparent to a person of ordinary skill in the art that such specific descriptions are merely desirable embodiments and that the scope of the present disclosure is not limited thereby. Accordingly, the substantial scope of the present disclosure is defined by the appended claims and their equivalents.
[Accession No.]
Depository Name: Korea Center for Biological Resources (KCTC)
Accession No.: KCTC15485BP
Deposit Date: 20230626

1. An attenuated Salmonella strain transformed with a recombinant expression vector comprising an flgM gene and a melittin gene.

2. The attenuated Salmonella strain of claim 1, wherein the flgM gene and the melittin gene are linked via a linker.

3. The attenuated Salmonella strain of claim 1, wherein the recombinant expression vector further comprises an flhDC gene.

4. The attenuated Salmonella strain of claim 1, wherein the attenuated Salmonella strain has a loss of function of one or more genes selected from the group consisting of aroA, aroC, aroD, aroE, asd, Rpur, htrA, ompR, ompF, ompC, galE, cya, crp, cyp, phoP, phoQ, rfaY, dksA, hupA, sipC, clpB, clpP, clpX, pab, nadA, pncB, pmi, rpsL, hemA, rfc, poxA, galU, cdt, pur, ssa, guaA, guaB, fliD, flgK, flgL, relA, and spoT.

5. The attenuated Salmonella strain of claim 1, wherein the attenuated Salmonella strain has an enhanced ability to secrete melittin.

6. The attenuated Salmonella strain of claim 1, wherein the attenuated Salmonella strain is deposited under Accession No. KCTC15485BP.

7. A pharmaceutical composition for anti-cancer use, comprising the attenuated Salmonella strain of claim 1 as an active ingredient.

8. A recombinant expression vector comprising: an flgM gene; and a melittin gene.

9. The recombinant expression vector of claim 8, wherein the flgM gene and the melittin gene are linked via a linker.

10. The recombinant expression vector of claim 8, further comprising an flhDC gene.

11. The recombinant expression vector of claim 8, wherein the flgM gene consists of a nucleotide sequence of SEQ ID NO: 1.

12. The recombinant expression vector of claim 8, wherein the melittin gene consists of a nucleotide sequence of SEQ ID NO: 2.

13. The recombinant expression vector of claim 10, wherein the flgM gene; the melittin gene; and the flhDC gene are expressed under the control of the same inducible promoter.
